(19) United States
(12) Patent Application Publication
(10) Pub. No.: US 2021/0341497 A1
(43) Pub. Date: Nov. 4, 2021

(54) DIAGNOSIS METHOD FOR DISEASES ACCOMPANIED BY REDUCED BONE DENSITY

(71) Applicant: GEOMAR HELMHOLTZ ZENTRUM FÜR OZEANFORSCHUNG KIEL, Kiel (DE)

(72) Inventor: Anton EISENHAUER, Heikendorf (DE)

(73) Assignee: GEOMAR Helmholtz-Zentrum fuer Ozeanforschung Kiel, Kiel (DE)

(21) Appl. No.: 17/272,012

(22) PCT Filed: Aug. 28, 2019
(86) PCT No.: PCT/EP2019/072951

§ 371 Date: Feb. 26, 2021

(30) Foreign Application Priority Data
Aug. 29, 2018 (DE) 10 2018 214 660.8
Aug. 29, 2018 (EP) 18191474.8

(51) Int. Cl. G01N 33/84

(52) U.S. Cl. CPC G01N 33/84; G01N 2800/10

(57) ABSTRACT

The invention relates to a method for diagnosing a disease that is accompanied by reduced bone density and/or calcium loss, comprising the following steps: a) determining isotope ratios or amount ratios of alkaline-earth metal portions in a sample of urine or blood or stool, b) thereby determining at least one sample value, and c) comparing the at least one sample value with a threshold value, the threshold value being selected independently of one or more factors determined by the individual patient. The invention further relates to a test kit for use in carrying out the method.

The invention relates to the diagnosis and/or the exclusion of diseases accompanied by reduced bone density, such as osteoporosis, osteomalacia, multiple myeloma and/or renal dysfunction.

Diseases accompanied by reduced bone density, such as osteoporosis, osteomalacia, multiple myeloma and/or renal dysfunction are a big problem throughout the world. According to WHO, osteoporosis alone features among the 10 most frequent diseases.

Osteoporosis occurs increasingly from a certain age and occurs approximately four times as frequently in women as in men.

In Germany, a rise of more than 20% was recorded between 2010 and 2016 in the principal diagnoses of osteoporosis with pathological fracture.

It therefore appears to be very expedient, in particular in the relevant risk group, to perform diagnoses increasingly (mass screening) in order to detect the risk of developing osteoporosis early on and to take counteractive measures. The risk of undetected osteoporosis and the possible resulting health consequences (bone breaks, postural defects) could also thus be minimised.

In view of the high number of diseases, a screening process that could be used within the scope of preventative medicine and can therefore separate unaffected patients from the possibly affected patients would also be very desirable.

It would also be very expedient to perform diagnoses accompanied by therapy in order to detect a therapy that is not progressing as desired against diseases accompanied by reduced bone density and, in an ideal situation, to also be able to learn more about the causes of the disease and thus take more suitable measures.

The currently used standard method for diagnosing and monitoring osteoporosis and other bone metabolism disorders with increased risk of bone break is X-ray based DXA or DEXA (dual-energy X-ray absorptiometry). A disadvantage here is the resultant radiation exposure for the patient and the poor comparability of the determined values; the results therefore do not provide any absolute values, and instead only a deviation from the age- and gender-specific average is determined and compared with the standard deviation.

US 2013/0115650 A1 proposes using the ratio of ⁴⁴Ca/⁴²Ca as a biomarker for diagnosing diseases accompanied by a change to the mineral balance of the human skeleton. However, in order to arrive at a conclusion, a baseline for each individual must first be determined (first sample), and shortly after starting therapy a further sample must be taken and measured.

Skulan, Joseph; at el; “Natural Calcium Isotopic Composition of Urine as a Marker of Bone Mineral Balance” Clinical Chemistry, 53, 1155-1158 (2007) propose measuring the calcium-isotope ratio δ⁴⁴/⁴⁰Ca in the urine at different times over a longer period in order to monitor the mineral balance of the skeleton and detect any possible “bone atrophy” caused by a longer period of being confined to bed.

US 2014/0273248 al relates to the monitoring the mineral balance of the human skeleton in cancer patients. Here too, a baseline must first be determined, then, once therapy has begun, consisting of the administration of aromatase inhibitors, further samples are taken.

Gordon et al., “predicting multiple myeloma disease activity by analyzing natural calcium isotopic composition”, Leukemia, 28, 2112-2115, (2014) attempt to detect cancer of the haematopoietic system on the basis of the calcium isotope composition of the blood. In the event of a change in status from “non-active disease” to “active disease”, it was not possible to determine any change to the calcium isotope values; the authors attribute this to unknown factors which are dependent, amongst other things, on the individual condition of the patient, and which conceal the solitary influence of the multiple myeloma and thus also reduce the sensitivity and specificity of the measured values. Diagnosis on the basis of a measured value is not possible according to Gordon et al.

Diagnosis methods which require a standardisation to the individual patient by means of multiple measurements (for example by recording a baseline) tend to be unsuitable for use in preventative medicine, in particular also within the scope of a screening process.

The object of the invention is therefore to provide a method for diagnosing diseases which are accompanied by reduced bone density, in which method it is possible to dispense with X-ray radiation, which subjects the patient to an exposure risk, and with a complex standardisation to the individual patient, for example by means of a baseline.

The object of the invention is achieved by a method for diagnosing a disease that is accompanied by reduced bone density and/or calcium loss, comprising the following steps:
a) determining isotope ratios or amount ratios of alkaline-earth metal portions in a sample of urine or blood or stool,
b) thereby determining at least one sample value, and
c) comparing the at least one sample value with a threshold value, wherein the threshold value is selected independently of one or more factors determined by the individual patient.

The sample values are determined here in previously taken blood and/or urine and/or stool samples.

The expression that the threshold value is selected independently of one or more factors conditional on the individual patient shall be understood within the scope of the present invention in particular such that a patient-related baseline regarding the sample values to be ascertained does not have to be determined beforehand, nor do further factors, such as a nutrition plan to be followed prior to the actual diagnosis or similar factors or measurements of previous values of the individual patient, have to be performed or included in the diagnosis method. In other words, the threshold value, depending on the sample type of the urine, blood or stool sample, is a fixed numerical value. The threshold value is in particular not simply a mean value from a plurality of samples, and instead it was surprisingly possible, within the scope of the invention, to determine a fixed threshold value by far-reaching studies lasting many years and by validations for each sample type.

Advantageously, a method for diagnosing diseases accompanied by reduced bone density and/or calcium loss which is both simplified significantly in respect of its handling and can thus also be used as a preventative measure can now be provided for the first time. Due to the omission according to the invention of prior determinations of baselines and other influencing factors, such as diet monitoring and food diaries, which previously had to be performed and provided by the individual patients even before the actual diagnosis was made, the execution and application of the method are significantly simplified, which allows a broad distribution and can be used in preventative diagnosis, especially for older women, also without any prior suspicion. A significant cost reduction for the simplified method is additionally a key factor in the further application and distribution, and therefore such a “screening” is made available for the first time for a much larger patient group.

The method according to the invention, the validity and significance of which will also be explained in greater detail further below, has also been able to overcome a prejudice prevailing in particular as a result of the specialist article by Gordon et al. in Leukemia, 2014, which ultimately considered the simultaneous consideration of individual patient parameters to be essential to the evaluations of the isotope ratios of the alkaline-earth metals in the samples. This is because the consideration of a group of patients with multiple myeloma and their isotope ratios δ^{44/42}Ca without these individual patient parameters, as asserted by the authors themselves, does not lead to a reliable diagnosis method. In addition, no diagnosis-relevant threshold value is specified; the stated values are merely mean values formed from the samples of the groups concerned.

The sample values are ascertained isotope ratios or amount ratios of alkaline-earth elements. In particular, the values are isotope ratios of calcium (Ca) or the amount ratio of alkaline-earth elements of calcium (Ca) to strontium (Sr).

The sample values are preferably isotope ratios or amount ratios of alkaline-earth elements ascertained by mass spectroscopy.

In a preferred embodiment of the method, the isotope ratios of calcium (Ca) are determined in step a).

In a further preferred embodiment of the method, the amount ratio of the alkaline-earth elements of calcium (Ca) to strontium (Sr) is determined in step a).

In one aspect of the invention, a fixed threshold value which is independent of the individual and which was defined depending on the sample type is used in step c) as a comparison value.

In one aspect of the invention, the threshold value in step c) for the isotope ratio ⁴⁴Ca/⁴²Ca in a blood sample according to a δ notation is δ^{44/42}Ca_{blood}=−0.85±0.0600.

In another aspect of the invention, the threshold value in step c) for the isotope ratio ⁴⁴Ca/⁴² Ca in a urine sample according to a δ notation is δ^{44/42}Caᵤᵣᵢₙₑ=0.23±0.06%.

The threshold value, corresponding to formula:

$${δ}^{m 3 / m 2} Ca = {δ}^{m 3 / m 1} Ca \times [ \frac{{ln}^{( m 3 / m 2 )}}{{ln}^{( m 3 / m 1 )}} ]$$

with m3>m2>m1, wherein m3, m2, m1 represent the mass numbers, can be transferred to other isotope ratios of calcium.

In a further aspect of the invention, the threshold value in step c) for the amount ratio of alkaline-earth elements of calcium (Ca) to strontium (Sr) is 1772±250 mol_{Ca}/mol_{Sr}.

In a preferred embodiment of the invention, in step b) one sample value is ascertained as δ^{44/42}Caᵤᵣᵢₙₑ and a further sample value is ascertained as δ^{44/42}Ca_{blood} and in step c) the difference between the two sample values is compared with a threshold value in order to additionally ascertain the kidney function.

In a further preferred embodiment of the invention, in step b) one sample value is determined as δ^{44/42}Caₛₜₒₒₗ and a further sample value is determined as δ^{44/42}Ca_{blood} and in step c) the difference between the two sample values is compared with a threshold value in order to additionally ascertain the intestinal function.

In one aspect of the invention, the disease accompanied by reduced bone density and/or calcium loss is osteoporosis, osteomalacia, multiple myeloma and/or renal dysfunction.

In a preferred embodiment of the method, the amount ratios in step a) are determined as molar ratios of the alkaline-earth metal portions in the sample or as mass ratios of the alkaline-earth metal portions in the sample.

A further preferred embodiment of the method is characterised in that if in step b) a sample value is determined as δ^{44/42}Caᵤᵣᵢₙₑ and in step c) the comparison with the threshold value according to a δ notation of δ^{44/42}Caᵤᵣᵢₙₑ=0.23±0.06% reveals that the sample value is greater, the presence of an osteoporosis disease can be excluded as unlikely.

A further preferred embodiment of the method is characterised in that if in step b) a sample value is determined as δ^{44/42}Ca_{blood} and in step c) the comparison with the threshold value according to a δ notation of δ^{44/42} Ca_{blood}=−0.85±0.06% reveals that the sample value is greater, the presence of an osteoporosis disease can be excluded as unlikely.

In one aspect of the invention the method is a test in which, by measuring the isotope ratio of calcium isotopes, it is possible to determine whether, in post-menopausal women, there are no diseases accompanied by a reduced bone density, such as osteoporosis, osteomalacia and/or multiple myeloma or whether there is an increased likelihood for a positive diagnosis of one of these diseases.

In a further aspect of the invention, the isotope ratio ⁴⁴Ca/⁴²Ca in a previously removed blood sample is determined and is standardised in accordance with a δ notation to an international standard.

The δ^{m3/m2}Ca values can also be transferred, in accordance with the following formula, to other Ca isotope ratios (for example with the inclusion of ⁴⁰Ca, ⁴¹Ca, ⁴⁶Ca, ⁴⁸Ca, ⁴³Ca):

$${δ}^{m 3 / m 2} C a = {δ}^{m 3 / m 1} C a \times [ \frac{{ln}^{( m 3 / m 2 )}}{{ln}^{( m 3 / m 1 )}} ]$$

with m3>m2>m1.

In accordance with the invention, the value thus ascertained is compared with a threshold value (for δ^{44/42}Ca_{blood}: −0.85±0.06%); if the values lie above this threshold value, there are no diseases accompanied by a reduced bone density, such as osteoporosis, osteomalacia and/or multiple myeloma, and if the values lie below the threshold value, there is an increased risk of a positive diagnosis of one of the diseases accompanied by a reduced bone density.

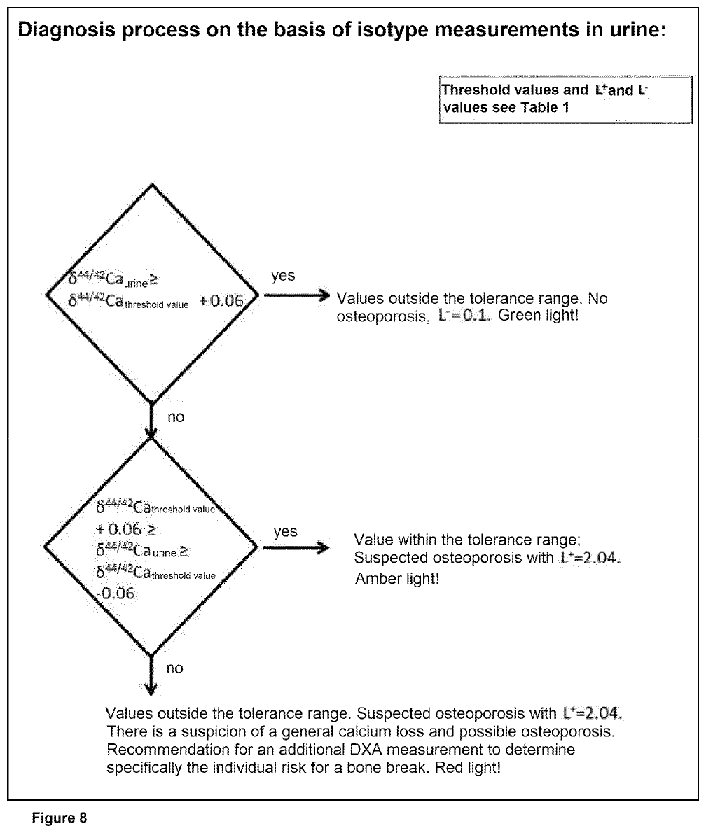

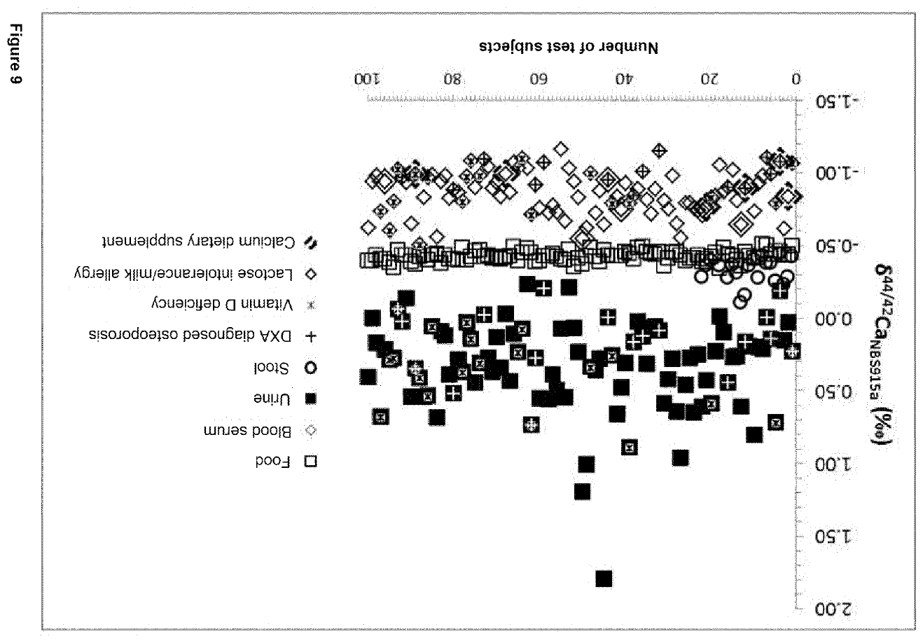

It is therefore possible to come to a diagnosis result on the basis of a single sample. The threshold value of δ^{44/42}Ca_{blood}=−0.85±0.06% was ascertained on the basis of a registered study (NCT02967978) of 100 post-menopausal female test subjects who were between 50 and 75 years old in accordance with the Helsinki criteria “for good clinical practice” (Osteogeo study). Due to the variation of measured values, the value should be provided with a statistical uncertainty of ±0.06%, i.e. from −0.79% to −0.91% there is an uncertainty, resulting from the measuring method, as to whether or not the patient is suffering from bone atrophy. The latter should be taken into consideration in the assessment or diagnosis (see FIGS. 8, 9 and 10).

The threshold value for δ^{44/42}Ca_{blood} can also be transferred on the basis of the following formula to other Ca isotope ratios (for example with the inclusion of ⁴⁰Ca, ⁴¹Ca, ⁴⁶Ca, ⁴⁸Ca, ⁴³Ca):

$${δ}^{m 3 / m 2} C a = {δ}^{m 3 / m 1} C a \times [ \frac{{ln}^{( m 3 / m 2 )}}{{ln}^{( m 3 / m 1 )}} ]$$

with m3>m2>m1.

The result is very reliable: the sensitivity of the method is 94.4%, the negative likelihood factor is 0.1; this means that women whose measured values lie above the threshold value have, relative to a healthy person, only a 0.1 times likelihood of a positive osteoporosis diagnosis.

Prior to the study, it was not anticipated that a diagnosis could be possible only on the basis of one value and with this validity; rather, it had to be assumed that the calcium isotope content of food eaten would also have an influence on the result. This would mean that a standardisation to the individual person would be necessary by measuring various samples from a person and/or possibly by taking a number of measurements when a diet is followed which controls the calcium isotope content of the food.

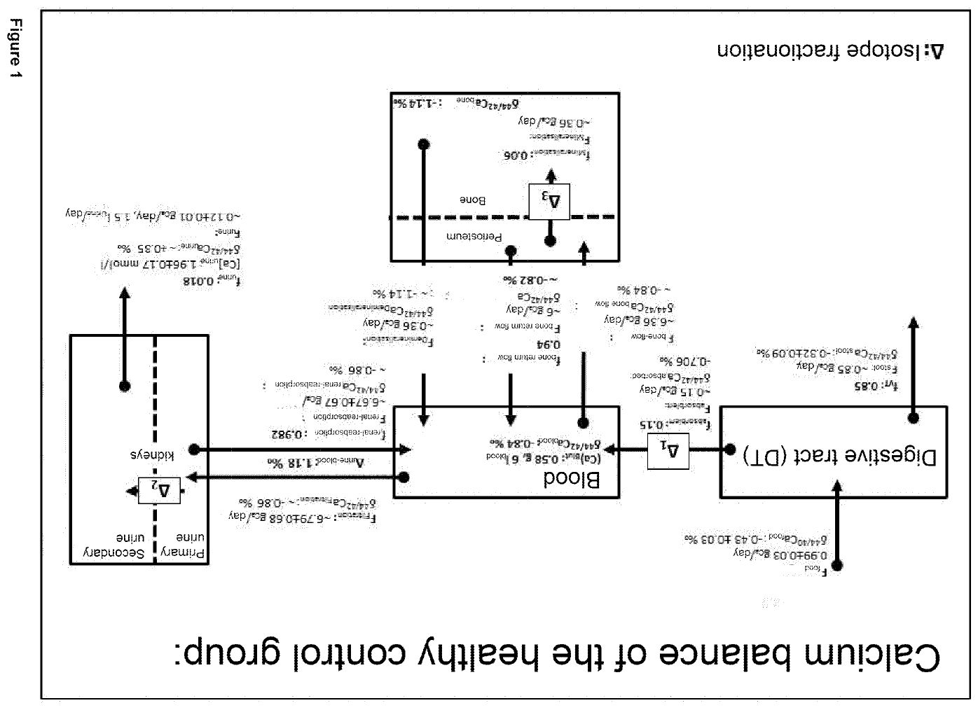

FIG. 1 shows the measured calcium isotope in the food, stool blood and urine of the 100 post-menopausal women from the Osteogeo study. Although the dietary behaviour of the 100 women fluctuated very greatly from individual to individual, their calcium isotope content was very constant and can be estimated at δ⁴⁴Ca/⁴²Ca: −0.43±0.05% within narrow limits. As a result, it also follows that a detailed questionnaire regarding the food eaten is not absolutely necessary to a diagnosis, since a generally known value can be used as a starting point, even if there is a particular dietary situation present, such as vegetarianism or veganism.

Furthermore, it was possible to show by the Osteogeo study that the influence of the calcium from the food on the calcium isotope composition of the blood is low, since the absorption rate of calcium from the intestine is at most 30% to approximately 10%. In other words, of the daily ingested calcium in the order of approximately 1 gram of calcium per day (g_{Ca}/day), only approximately 0.3 to 0.15 g is absorbed. This corresponds, however, only to approximately 2.2% to 1.1% of the amount of calcium introduced daily into the bloodstream (13.18 g Ca/day) from the kidneys (approximately 6.67 g Ca/day, approximately 50%) and from the bones (6.36 g Ca/day, approximately 48%). It follows that any isotope variations of the calcium in the food are low to negligible due to the mass balance.

Although the calcium isotope of the food for all test subjects in the study was practically constant, the calcium isotope values for blood, urine and stool are different between the various organs on account of endogenous fractionation processes.

The calcium flows between the various organs in healthy post-menopausal women as ascertained within the scope of the Osteogeo study can be shown graphically hereinafter: see FIG. 1.

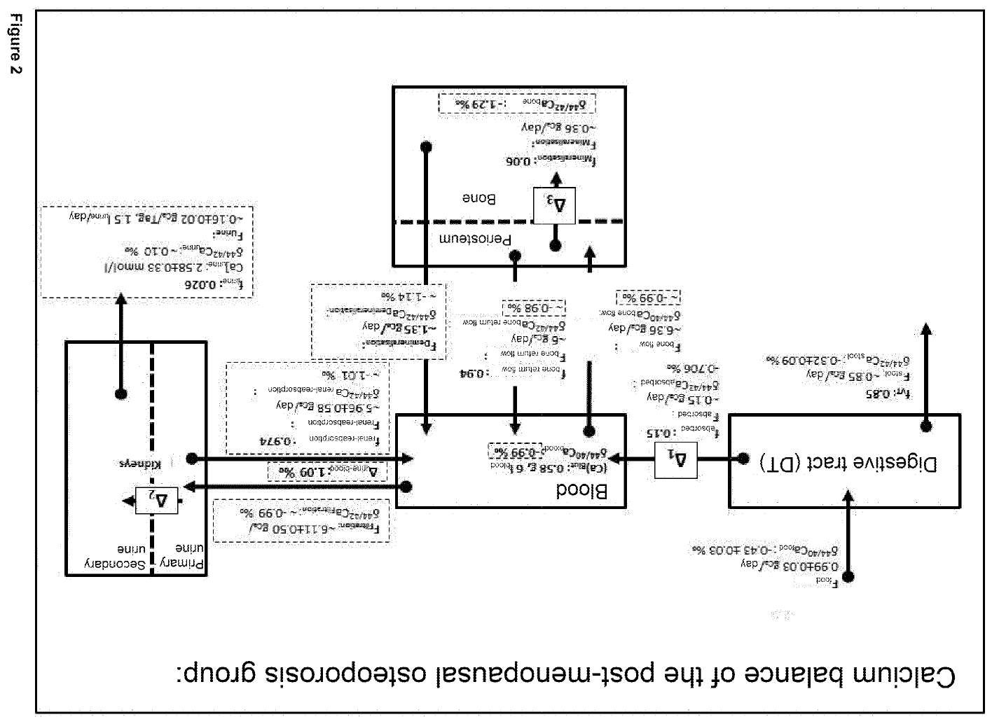

The following drawing according to FIG. 2 shows the calcium flows, also ascertained, between the organs in post-menopausal women suffering from osteoporosis. The differences in the calcium flows between the involved compartments are marked by means of a broken line.

The invention has now identified that these profound findings regarding the calcium flows between the organs in healthy women and those suffering from osteoporosis can be implemented in a test method in which it is possible to test for the absence of a disease accompanied by reduced bone density, such as osteoporosis, osteomalacia and/or multiple myeloma (negative test), and at the same time for an increased likelihood of a positive diagnosis of said diseases being made. It is therefore possible to arrive at a diagnosis result on the basis of a single blood sample.

A further aspect of the invention relates to a test in which, by measuring the isotope ratio of calcium isotopes in a previously taken urine sample, it is possible to determine whether in post-menopausal women there is no disease accompanied by reduced bone density, such as osteoporosis, osteomalacia and/or multiple myeloma, or whether there is an increased likelihood for a positive diagnosis of one of these diseases.

The isotope ratio ⁴⁴Ca/⁴²Ca is determined in a previously taken urine sample and is standardised in accordance with a δ notation to an international standard.

In accordance with the invention, the value thus ascertained is compared with a threshold value (for δ⁴⁴Ca/⁴²Caᵤᵣᵢₙₑ 0.23±0.06%); if the values lie above this threshold value, osteoporosis is present, if the values lie below the threshold value, there is an increased risk of a positive osteoporosis diagnosis.

It is therefore possible to arrive at a diagnosis result on the basis of a single sample. The threshold value of δ⁴⁴Ca/⁴²Caᵤᵣᵢₙₑ 0.23±0.06% was ascertained on the basis of a study of the 100 female test subjects aged between 50 and 75 years.

The threshold value δ⁴⁴Ca/⁴²Caᵤᵣᵢₙₑ can also be transferred on the basis of the following formula to other Ca isotope ratios (for example with the inclusion of ⁴⁰Ca, ⁴¹Ca, ⁴⁶Ca, ⁴⁸Ca, ⁴³Ca):

$${δ}^{m 3 / m 2} C a = {δ}^{m 3 / m 1} C a \times [ \frac{{ln}^{( m 3 / m 2 )}}{{ln}^{( m 3 / m 1 )}} ]$$

with m3>m2>m1.

The sensitivity of the method is 72.2%, the negative likelihood factor is 0.41; this means that women whose measured values lie above the threshold value have, relative to a healthy person, only a 0.41 times likelihood of a positive osteoporosis diagnosis.

Prior to the study, it was not anticipated that a diagnosis could be possible only on the basis of one value; rather, it had to be assumed that the calcium isotope content of food eaten would also have an influence on the result. This would mean that a standardisation to the individual person would be necessary by measuring various samples from a person and/or possibly by taking a number of measurements when a diet is followed which controls the calcium isotope content of the food.

The invention has now identified that these profound findings regarding the calcium flows between the organs in healthy women and those suffering from osteoporosis can be implemented in a test method in which it is possible to test for the absence of a disease accompanied by reduced bone density, such as osteoporosis, osteomalacia and/or multiple myeloma (negative test), and at the same time for an increased likelihood of a positive diagnosis of said diseases being made. It is therefore possible to arrive at a diagnosis result on the basis of a single urine sample.

The possibility of the diagnosis on the basis of a single sample of an individual is a very surprising result. The existence of the threshold value of δ⁴⁴Ca/⁴²Caᵤᵣᵢₙₑ 0.23±0.06% and δ⁴⁴Ca/⁴²Ca_{blood -}0.85±0.06% was not expected.

A further aspect of the invention relates to a test in which, by measuring the ratio of the alkaline-earth elements calcium (Ca) to strontium (Sr) in urine (Ca/Sr)ᵤᵣᵢₙₑ, it is possible to characterise the resorption capability of Ca and therefore the likelihood of the presence of a disease accompanied by reduced bone density, such as osteoporosis, osteomalacia and/or multiple myeloma. The result is surprising in particular against the background that the ratio of Ca and magnesium (Mg), which is likewise an alkaline-earth element, does not have this correlation.

A statistically significant relationship of the (Ca/Sr)ᵤᵣᵢₙₑ values with the DXA values could be ascertained from the ROC (receiver-operating characteristics) analysis. The DXA method is the currently valid standard method for ascertaining bone density and for diagnosing osteoporosis.

A (Ca/Sr)ᵤᵣᵢₙₑ threshold value could be ascertained which makes it possible to distinguish a female patient suffering from osteoporosis from healthy female patients. The threshold value was 1772±250 mol_{Ca}/mol_{Sr} and separated patients with disease from healthy patients.

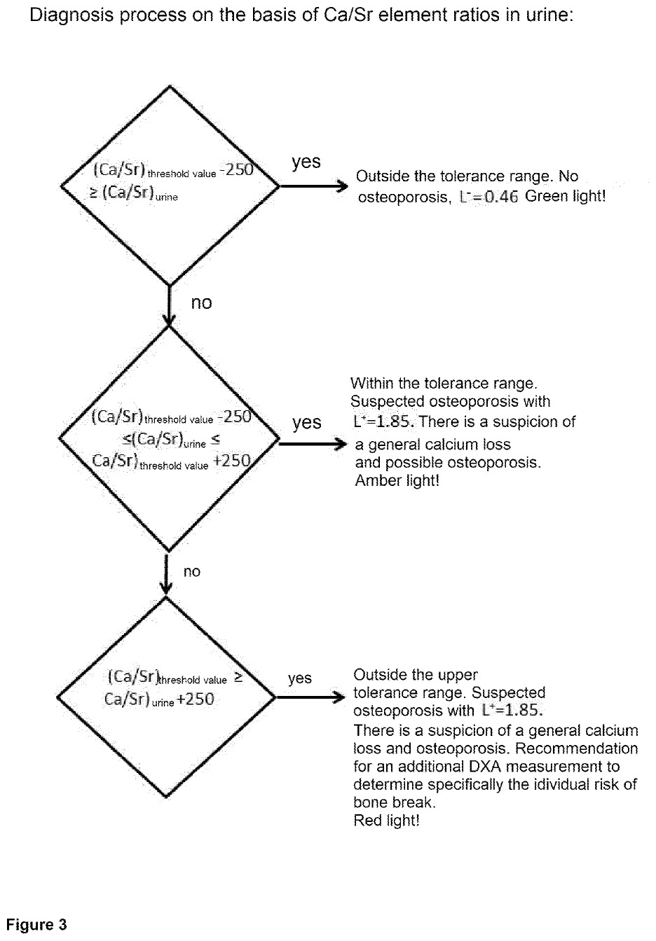

The diagnosis process can be seen in graphically hereinafter according to FIG. 3.

A further aspect of the invention relates to a method for determining kidney function on the basis of the comparison of the Ca isotope difference in a previously taken urine and blood sample.

It has been found that the Ca isotope difference Δ_{urine-blood}=δ^{44/42}Ca_{blood} reflects the recycling efficiency of the kidneys for trace elements and that this relationship is non-linear, but is calculable insofar as the recycling rate f_{renal-absorption} of the kidneys can be calculated from the values (see the figures).

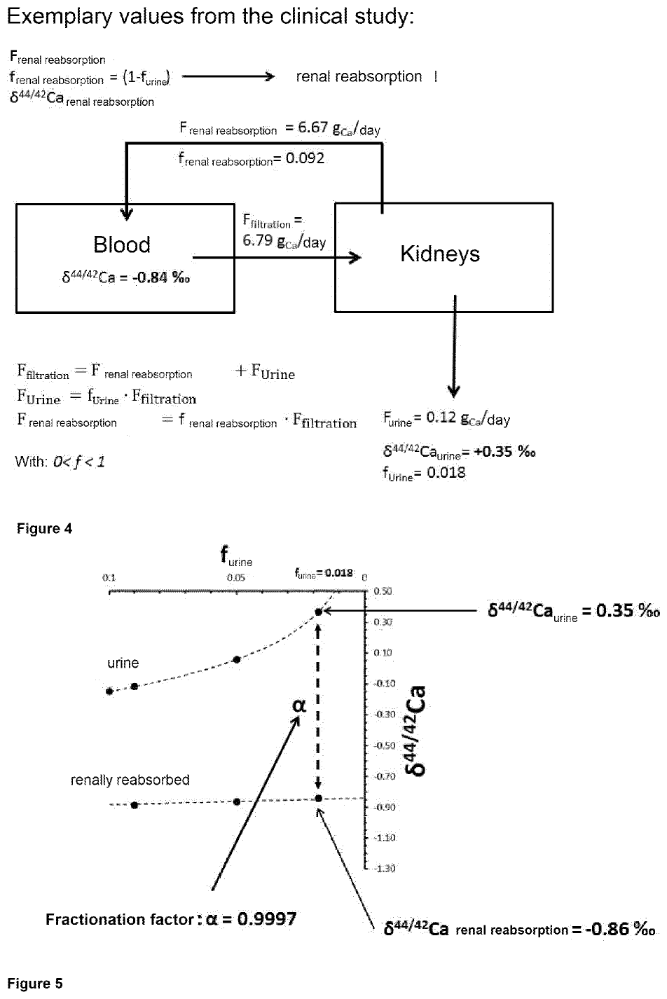

The following drawing according to FIG. 4 shows the calcium flows and isotope fractionation between the blood system and the kidneys. With the aid of the calcium isotope ratios measured in blood and urine, the rate of the renal reabsorption of calcium in the kidneys can be calculated. The latter is a measure for the functionality of the kidneys.

The functionality of the kidneys can then be classified in accordance with the invention on the basis of the calculated recycling rate in accordance with Table 1:

TABLE 1
classification (threshold values) of the kidney function
by means of renal reabsorption (f_{renal-absorption})

1.00 > f_{renal-absorption} = ≥0.98 −> very good kidney function
0.98 > f_{renal-absorption} = ≥0.95 −> good kidney function
0.95 > f_{renal-absorption} = ≥0.92 −> kidney dysfunction
0.92 > f_{renal-absorption} −> increasing kidney dysfunction

The values were calibrated here by determining the eGFR (estimated Glomerular Filtration Rate), which was performed on female patients having different kidney function. With regard to the glomerular filtration rate (GFR) it must be said that this is the currently established method for estimating kidney function.

The GFR is either determined in a relatively complex manner from 24-h urine collection as creatinine clearance or by means of a wide range of different eGFR approximation formulas from the plasma creatinine concentration under consideration of the skin colour, age, gender and also other variables.

What is known as the MDMR formula is the most common [Saulo Klahr, Andrew S. Levey, Gerald J. Beck, Arlene W. Caggiula, Lawrence Hunsicker, John W. Kusek, Gary Striker, The Modification of Diet in Renal Disease Study Group: The Effects of Dietary Protein Restriction and Blood-Pressure Control on the Progression of Chronic Renal Disease. In: N Engl J Med. 330, Nr. 13, 31 Mar. 1994, pages 887-884. doi:10.1056/NEJM199403313301301].

The approximation formulas are validated for ambulant patients suffering from chronic kidney disease with moderate to severe kidney function limitation (stage 3 and 4). The formulas are generally not applicable for minor kidney dysfunction or also no kidney dysfunction.

The formulas are also not suitable for determining the glomerular filtration rate in people with:
normal kidney function
minor kidney dysfunction.
In particular, in people with a glomerular filtration rate above 60 ml/min, the MDRD formula underestimates this by approximately 10 ml/min [Stevens, Lesley A. et al.: Evaluation of the Modification of Diet in Renal Disease Study Equation in a Large Diverse Population. In:J Am Soc Nephrol. Nr. 18, 2007, pages 2749-2757 (asnjournals.org)]
acute kidney function deterioration,
severe obesity
heavily reduced muscle mass (amputation of limbs, undernourishment)
with particularly high muscle mass (dietary supplements in bodybuilders)
low creatine supply with food (vegetarian).
for monitoring the kidney function in the particularly important early stage of diabetic nephropathy [Nephrology beyond JASN. Eberhard Ritz feature Editor: Estimated GFR: Are There Limits to Its Utility? J Am Soc Nephrol, 2006, 17, pages 2077-2085]

In accordance with the invention the Ca isotope difference Δ_{urine-blood}=δ44/42Caᵤᵣᵢₙₑ−δ44/42Ca_{blood} is used as a basis to classify the recycling efficiency of the kidneys. The recycling rate f_{renal-absorption} of the kidneys is calculated from the values. The classification is then performed on the basis of the threshold values from Table 1.

The δ notation δ^{44/42}Ca can also be transferred here on the basis of the following formula to other Ca isotope ratios (for example with inclusion of ⁴⁰Ca, ⁴¹Ca, ⁴⁶Ca, ⁴⁸Ca, ⁴³Ca):

$${δ}^{m 3 / m 2} C a = {δ}^{m 3 / m 1} C a \times [ \frac{{ln}^{( m 3 / m 2 )}}{{ln}^{( m 3 / m 1 )}} ]$$

with m3>m2>m1.

The variables relevant for the calculation δ^{44/42}Ca_{blood}, δ^{44/42}Caᵤᵣᵢₙₑ and Fᵤᵣᵢₙₑ are known by measurement. Fᵤᵣᵢₙₑ specifies the daily loss of calcium via the urine.

The Ca isotopes and flows marked by broken lines can be calculated by the Rayleigh equations and the isotope balance. The following approach is assumed:

The definition of the δ notation is:

$${δ}^{44 / 42} C {a}_{blood} ( \% 0 ) = [ \frac{{( \frac{{44}_{Ca}}{{42}_{Ca}} )}_{b l o o d}}{{( \frac{{44}_{Ca}}{{42}_{Ca}} )}_{s tandard}} - 1 ] \qquad ( 01 )$$

and for urine

$${δ}^{44 / 42} C {a}_{u r i n e} ( \% 0 ) = [ \frac{{( \frac{{44}_{Ca}}{{42}_{Ca}} )}_{urine}}{{( \frac{{44}_{Ca}}{{42}_{Ca}} )}_{s tandard}} - 1 ] \qquad ( 02 )$$

For simplification:

$${R}_{blood} = {( \frac{4 {4}_{C a}}{4 {2}_{C a}} )}_{blood}$$

$$and$$

$${R}_{u r i n e} = {( \frac{4 {4}_{C a}}{4 {2}_{C a}} )}_{u r i n e}$$

The ratio of Rᵤᵣᵢₙₑ/R_{blood} is then given from equation (03):

$$\frac{{R}_{urine}}{{R}_{b l o o d}} = \frac{{( \frac{{44}_{Ca}}{{42}_{Ca}} )}_{urine}}{{( \frac{{44}_{Ca}}{{42}_{Ca}} )}_{b l o o d}} = \frac{{δ}^{44 / 42} {Ca}_{urine} - 1}{{δ}^{44 / 42} {Ca}_{b l o o d} - 1} \qquad ( 03 )$$

Equation (03) also has the advantage that the ratio of Rᵤᵣᵢₙₑ/R_{blood} can be calculated without knowing the absolute (⁴⁴Ca/⁴²Ca) standard value.

To describe the sequestration of a Ca flow and the accompanying Ca isotope fractionation, the formula for the Rayleigh distillation will now be applied.

The following known (measured) variables are included in formulas (04) to (06) of the formulas describing the distillation:
a) the ratio of the measured Ca isotope values of the urine and blood

Rᵤᵣᵢₙₑ/R_{blood}=(δ^{44/42}Caᵤᵣᵢₙₑ−1)/(δ^{44/42}Ca_{blood}−1)

and the following unknown variables:
i. The distribution quotient that describes the division of the Ca between blood and urine (0≤fᵤᵣᵢₙₑ≤1). The expression fᵤᵣᵢₙₑ describes the relative proportion of the calcium expelled with the urine and f_{renal-absorption}=(1−fᵤᵣᵢₙₑ) describes the proportion of the calcium that is reabsorbed in the bloodstream.
ii. the fractionation factor “α” between blood and urine
iii. and the Ca isotope value δ^{44/42}Ca_{renal-absorption} of the calcium fed back into the bloodstream.

The equations (04) to (06) are applied.

$$\begin{matrix} \frac{{R}_{urine}}{{R}_{b l o o d}} = {furine}^{( α - 1 )} & ( 04 ) \\ \frac{{R}_{renal - absorption}}{{R}_{b l o o d}} = \frac{{δ}^{44 / 42} C {a}_{renal - absorption}}{{δ}^{44 / 42} C {a}_{b l o o d} - 1} = \frac{( 1 - {f}_{{urine}^{α}} )}{1 - f} & ( 05 ) \\ {δ}^{44 / 42} C {a}_{b l o o d} = {f}_{urine} \cdot {δ}^{44 / 42} C {a}_{urine} + ( 1 - {f}_{urine} ) \cdot {δ}^{44 / 42} C {a}_{renal - absorption} & ( 06 )\end{matrix}$$

For further simplification and handling of equations (04) to (06), what is commonly known as the “small approximation” in the field of mathematics can be used. The advantage of this approach is that the measured isotope difference between blood and urine (Δ_{urine-blood}=δ^{44/42}Caᵤᵣᵢₙₑ−δ^{44/42}Ca_{blood}) can be used. Furthermore, a direct relationship between the isotope urine-blood difference and the filtration efficiency “f” can then be produced:

It is (“small approximation”):

$$ln ( \frac{{R}_{u r i n e}}{{R}_{blood}} ) ≅ \frac{{δ}^{44 / 42} C {a}_{u r i n e} - {δ}^{44 / 42} C {a}_{blood}}{1000} ≅ {Δ}_{u rine - blood}$$

The small approximation for ln(x)=x applies when x<<1. In isotope chemistry this is used for all ranges from approximately −30 to +40%. In that case, 1000 ln (x)=d¹lₐ−d¹l_{b} (d¹lₐ, d¹l_{b} isotope ratios). It is necessary to multiply by 1000 since the d¹lₐ are specified in per mille (%).

$$\frac{{R}_{urine}}{{R}_{b l o o d}} ≅ {e}^{\frac{{Δ}_{urine - bl o o d}}{1000}} ;$$

This results in:

Use in equation (04) gives:

$$\frac{{R}_{urine}}{{R}_{b l o o d}} = {e}^{\frac{{Δ}_{urine - bl o o d}}{1000}} = {f}_{urine}^{α - 1} ; \qquad ( 07 )$$

A similar approach is adopted for equation (05), so that then:

$$\frac{{R}_{renal - reabsorption}}{{R}_{b l o o d}} = {e}^{\frac{{Δ}_{( renal - reabsorption ) - b l o o d}}{1000}} = \frac{( 1 - furin {e}^{α} )}{1 - f} \qquad ( 08 )$$

The equation system to be solved is as follows:

$$\begin{matrix} {e}^{\frac{{Δ}_{urine - bl o o d}}{1000}} = {furine}^{( α - 1 )} & ( 09 ) \\ \frac{{δ}^{44 / 42} C {a}_{renal - absorption} - 1}{{δ}^{44 / 42} C {a}_{b l o o d} - 1} = \frac{( 1 - {furine}^{α} )}{1 - f} & ( 10 ) \\ {δ}^{44 / 42} C {a}_{blood} = {f}_{urine} \cdot {δ}^{44 / 42} C {a}_{urine} + ( 1 - {f}_{urine} ) \cdot {δ}^{44 / 42} C {a}_{renal - absorption} & ( 11 )\end{matrix}$$

The converted non-linear equation system (09) to (11) is identical to that in (04) to (06), but analytically in particular with equation (9) shows that there is a functional non-linear relationship between the Ca isotope difference between blood and urine and the recycling rate “f” of the kidneys. This equation system has three unknowns (f, α and δ^{44/42} Ca_{renal-reabsorption}) and can therefore be solved with the provided three equations. These, however, are a non-linear system, and therefore the rules of linear algebra do not apply here, and instead iterative optimisations have to be used when looking for a simultaneous solution.

The function values of equation (04) and (05) or, equally, (09) and (10) can be shown graphically. (Rayleigh equations; see FIG. 5).

The upper curve reflects the Ca isotope composition of urine (δ^{44/42} Caᵤᵣᵢₙₑ) as a function of the calcium remaining in the urine. The lower curve reflects the Ca isotope of the renally-reabsorbed (δ^{44/42}Ca_{renal-reabsorption}) calcium reabsorbed into the bloodstream. The fractionation factor “α” is responsible for the isotopic difference and its quantitative variable.

FIG. 2 graphically shows the relationship between the isotopic difference between blood and urine (Δ^{44/42}Ca_{blood-urine}) and the rate of renal absorption (f_{renal-absorption}). In addition, the ranges of kidney function (see further above) are shown.

In a further aspect, the invention relates to a method for determining the individual calcium (Ca) absorption from the intestine into the blood via the difference of the Ca isotope ratios δ^{44/42} Ca (delta notation). If osteoporosis is determined, the treating physician can thus be provided with important clues, in particular in conjunction with the findings regarding kidney function via the recycling efficiency of the Δ_{urine-blood}=δ^{44/42}Caᵤᵣᵢₙₑ−δ^{44/42}Ca_{blood}, as to which problems could be the cause of the osteoporosis and what therapy is advisable.

The determination is performed here on the basis of the difference of the calcium isotope ratios in the blood, stool and in the food. Here, a number of samples taken over a period of a few days are used.

The findings obtained from the Osteogeo study will be used to explain the calculation of the absorption rate via the intestinal blood barrier.

The δ^{44/42}Ca_{food} value measured within the scope of the study is −0.43±0.01%.

The measured value δ^{44/42}Caₛₜₒₒₗ value of the stool is −0.32±0.09%.

An unknown relative calcium amount (f_{blood-intestine}) is absorbed by the blood (f_{blood-intestine}:0≤f_{blood-intestine}≤1, wherein f_{blood-intestine} is referred to as the absorption rate), and a complementary Ca amount (1−f_{blood-intestine}) is expelled with the stool. As the Ca transfers from the intestine into the blood, there is an isotope fractionation of unknown magnitude (α), and therefore the Ca isotope value of the absorbed Ca (δ^{44/42}Ca_{absorbed}) is also unknown. The three unknown variables f_{blood-intestine}, α, and δ^{44/42}Ca_{absorbed} can be calculated by means of three non-linear equations:

The three unknown variables f_{blood-intestine}, α, and δ^{44/42}Ca_{absorbed} can be calculated from the three non-linear equations and the two measured, known isotope values (δ^{44/42}Ca_{food} and δ^{44/42}Caₛₜₒₒₗ)

$$\begin{matrix} \frac{{R}_{stool}}{{R}_{food}} = f blood - {intestine}^{( α - 1 )} & ( 01 ) \\ \frac{{R}_{absorbed}}{{R}_{food}} = \frac{( 1 - f blood - {intestine}^{α}}{1 - {f}_{blood - intestine}} & ( 02 ) \\ {δ}^{44 / 42} {Ca}_{food} = {f}_{blood - intestive} \cdot {δ}^{\frac{44}{42}} {Ca}_{stool} + ( 1 - {f}_{blood - intestine} ) \cdot {δ}^{44 / 42} {Ca}_{absorbed} & ( 03 )\end{matrix}$$

“R” is the measured ⁴⁴Ca/⁴²Ca ratio, which can be back-calculated from the δ notation:

$$It holds that \text{:} \frac{{δ}^{44 / 42} {Ca}_{stool}}{{δ}^{44 / 42} {Ca}_{food}} = \frac{{R}_{stool} - 1}{{R}_{food} - 1} and \frac{{δ}^{44 / 42} {Ca}_{absorbed}}{{δ}^{44 / 42} {Ca}_{food}} = \frac{{R}_{stool} - 1}{{R}_{food} - 1}$$

This, however, is a non-linear system, and therefore the rules of linear algebra do not apply here, and instead iterative optimisations have to be used when looking for a simultaneous solution. According to data from specialist medical literature [Heaney R P. The Calcium Economy. p 145-162, In: Weaver C M, Heaney R P, editors. Calcium in Human Health. Totowa, N.J.: Human Press; 2006], the average absorption rate f_{blood-intestine} is f_{blood-intestine}=0.3, i.e. 30%. In the cited passage from the literature it is shown that a test subject taking 20 mmol/d (0.8 g_{Ca}/day) with food and absorbing 3.4 mmol/d (0.136 g_{Ca}/day) from the Ca-containing digestive juices expels 16.6 mmol/d (0.66 g/_{Ca}/day). Taking into account only the food, the absorption is thus 17%, and taking into account the food and the Ca-containing digestive juices, the absorption is then approximately 30%.

A value of f_{blood-intestine}=0.3, i.e. 30%, can therefore actually be considered—under consideration of the Ca-containing digestive juices—as the target value with an average Ca absorption of approximately 1 g calcium.

The δ notation δ^{44/42}Ca can be transferred here, as in the previous inventions, on the basis of the following formula also to other Ca isotope ratios (for example with inclusion of ⁴⁰Ca, ⁴¹Ca, ⁴⁶Ca, ⁴⁸Ca, ⁴³Ca):

$${δ}^{m 3 / m 2} Ca = {δ}^{m 3 / m 1} Ca \times [ \frac{{ln}^{( m 3 / m 2 )}}{{ln}^{( m 3 / m 1 )}} ]$$

with m3>m2>m1.

As a result, the doctor thus discovers whether the osteoporosis present was triggered by renal osteopathy (kidney dysfunction) or by reduced calcium (Ca) absorption from the intestine, and therefore whether secondary osteoporosis is present.

### Material and Methods:

Without limiting the generality of the teaching, an exemplary approach will be explained hereinafter.

### Sample Removal for Blood and Urine:

A blood sample was taken from the patient by the doctor in the normal blood amount required for blood tests (approximately 8 ml). The blood sample was left to stand for half an hour and then centrifuged. The resulting blood serum was separated from the blood clot. For further chemical processing, only blood serum is used.

For further chemical processing, a blood amount is removed that corresponds to an absolute amount of 50 μg calcium.

The urine sample, approximately 10 ml, can be taken by the patient herself using a container provided for this purpose. For further chemical processing a urine amount is taken that, similarly to blood, corresponds to an absolute amount of 50 μg calcium.

Extraction of the Alkaline-Earth Elements from the Samples:

In a chemical process the calcium from the blood and the urine is extracted until a solution with a concentration of approximately 5 ppm is available for measurement y mass spectrometry.

The processing of the blood for chemical extraction of the calcium can be performed by the following method:

### Chemical Sample Processing to Determine Isotopes of Calcium in Blood and Urine

### Day 1

The vessels required for microwave digestion (MD) are filled with HNO₃ and H₂O₂.
The prepared samples and standards are pipetted into the corresponding vessel.
The vessels are sealed and the microwave apparatus is started and is set to MD for 1.5 hours.
The digested samples are recovered from the MD. During discharge, the solutions are filled one after the other into beakers and are placed on the heating plate to dry (overnight).

### Day 2

The digested samples dried overnight are incorporated in 1 ml HNO₃+0.5 ml H₂O₂ and sealed again and boiled for 3 hours.
The beakers are opened and the solution dried out.
The dried samples are incorporated in 1 ml 2 M HNO₃.

### Day 3

The concentration of the alkaline-earth elements strontium (Sr), calcium (Ca and magnesium) is measured on a Q-ICP-MS by means of standard methods.

### Day 4

In order to always have the same absolute Ca amounts (50 μg_{Ca}) for column chromatography, the amount of acid that is required for the automated measurement on an ESI PrepFast is calculated and the samples are diluted accordingly.
The samples are transferred from the beakers into the PrepFAST® tubes (Elemental Scientific).
the alkaline-earth elements are separated automatically by means of the PrepFAST® apparatus (Elemental Scientific).

### Day 5

The samples separated according to elements are transferred from the tubes back into the beakers and are placed on the heating plate to dry out.
Once dry, they are incorporated again in 1 ml HNO₃ and 0.5 ml H₂O₂ and are boiled sealed for a further 3 hours.

### Day 6

The beakers with the samples are opened and dried out.
The samples are incorporated in 10 ml HNO₃ and are left to stand for 4 hours to equilibrium.
The samples are transferred into the tubes for measurement by mass spectrometry on a Neptune® (plasma mass spectrometer, ThermoFisher) and are measured there.

In order to determine the isotope ratios and/or ratios of alkaline-earth elements, besides the mass spectrometry method, spectroscopy methods may also be used and utilise the mass dependency of the hyperfine structure of the spectral lines by means of light emission. Without limiting the general validity of the method, determination of the values by mass spectroscopy will be described hereinafter.

### Measurement by Mass Spectrometry:

This solution is generally measured in a plasma mass spectrometer (MC-ICP-MS:Multi-Collector-Ionisation Coupled mass spectrometer) (for example ThermoFisher, Neptune) (this method will be described briefly hereinafter), however, measurements may also be performed alternatively in a thermal ionisation mass spectrometer. The objective is to determine the calcium isotope composition in blood serum and/or urine. In the TIMS ⁴⁴Ca/⁴⁰Ca ratios and in the MC-ICP-MS ⁴⁴Ca/⁴⁰Ca ratios or alternative ratios are measured. Both isotope ratios give equivalent results. The values differ only by a factor:

⁴⁴Ca/⁴⁰Ca≅2.05·⁴⁴Ca/⁴⁰Ca.

Determination of the Calcium Isotope Composition ⁴⁴Ca/⁴⁰Ca with Use of a Plasma Mass Spectrometer:

Calcium isotope measurements are performed on an MC-ICPMS (Neptune®, Thermo Fisher Scientific). The mass spectrometer is equipped with nine Faraday beakers, eight of which are movable. These are designed such that the atomic masses (u) 42, 43, 43.5 and 44 can be measured simultaneously. In order to eliminate interfering Ca and Ar hydrides (for example ⁴⁰Ar₁H₂ on ⁴²Ca), an APEX IR sample introduction system (Elemental Scientific®) is used. All measurements are performed in medium resolution (m/Δm˜4000) on the interference-free plateau of the low-mass side of the central measurement peak. This is achieved by choosing a suitable medium beaker mass of 4.687±0.001 u and is verified daily.

The instrumental fractionation (“mass bias”) is corrected by applying the “standard sample-bracketing (SSB)” method. The measurement of a sample is corrected by measurements of a 5 μg/ml Ca solution produced from a 10000 μg/ml Ca-ICP standard solution. Each sample is measured at least four times during one session and the average value is used for the further procedure. The Ca isotope composition is specified as δ^{44/42}Ca in parts per thousand (%):

δ^{44/42}Ca (%)=[(δ^{44/42}Ca)ₛₐₘₚₗₑ/(δ^{44/42}Ca)_{reference}]−1

The measured Ca-ICP standard solution is used as primary reference material. The δ^{44/42}Ca_{ICP} values are then converted with use of the measured δ^{44/42}Ca_{ICP} values from NIST SRM 915a into NIST SRM 915a:

δ^{44/42}Ca_{SRM915a} (sample, %)=δ^{44/42}Ca_{ICP} (sample)−δ^{44/42}Ca_{ICP} (SRM 915a), i.e. all measured values are specified relative to the international standard SRM915a.

During each session, chemically unprocessed NIST SRM 915a material was measured at the start and at the end of each session, and these results were compared with those of chemically processed NIST SRM 915a. The average difference between processed and unprocessed SRM 915a should generally be less than 0.01%, and therefore the Ca-isotope fractionation during the chemical purification can be considered negligible.

For the background correction, the “On-Peak” approach is used. The measured intensities of a 1% HNO₃ solution are subtracted from the intensities of the subsequent sample measurements.

In addition, the measurements on residues of double-charged strontium (⁸⁴Sr, ⁸⁶Sr and ⁸⁸Sr) were examined in order to ensure the correct measurement of the intensities of the masses of ⁴²Ca, ⁴³Ca and ⁴⁴Ca. To this end, at the start of each session, the intensity ratios of the masses 42/43.5, 43/43.5 and 44/43.5 of a 2 μg/ml Sr solution were measured. These ratios were then used to calculate the intensities of double-charged Sr from the measured intensities of the mass 43.5 of a given sample.

For quality control, the δ^{44/42}Ca values of NIST SRM 1486 and IAPSO seawater standard measured during the measurement phase were compared with published values. The long-term reproducibility (2 SD=standard deviation) over a time period of approximately two months is generally better than 0.06% for all analysed reference materials.

For further quality assurance, a number of criteria were applied in order to reject of an individual measurement, an individual sample and entire sequences. An individual measurement or sequence is discarded if:

|δ^{44/42}Ca−2·δ^{43/42}Ca|>0.20%.
A sample measurement is discarded if the average intensity lies outside an intensity window of from 70 to 130%, compared with the average intensity of the 5 μg/ml Ca ICP solution or NIST SRM 915a solution of the same charge.
A complete sequence is rejected if more than one of the measured international reference materials is more than 0.2% removed from the literature value or the data do not fall along the mass-dependent fractionation line.

The Ca/Sr ratio is determined by means of a standard method on a quadrupole mass spectrometer (the method is described hereinafter).

### Determination of the Ca/Sr and Mg/Ca Element Ratios

Before the Ca isotope analysis, the Ca, Mg and Sr concentrations of the sample solutions are measured on a quadrupole mass spectrometer (Agilent 7500cx®) using a matrix-adapted external calibration curve and indium as internal standard. During the measurement, the intensity of the double-charged ions is kept below 2% in order to minimise interference with double-charged ⁸⁴Sr, ⁸⁶Sr and ⁸⁸Sr on ⁴²Ca, ⁴³Ca and ⁴⁴Ca. The entire blank of the solution is generally below 50 ng. The long-term reproducibility of the concentration measurements and their ratios using internationally available standards is generally better then 5% (1 SD=standard deviation).

### Evaluation of the Calcium Isotope Data:

The measured calcium isotope values were reported in the conventional δ notation:

$${δ}^{44 / 42} Ca = [ \frac{{( \frac{{44}_{Ca}}{{42}_{Ca}} )}_{sample}}{{( \frac{{44}_{Ca}}{{42}_{Ca}} )}_{standard}} - 1 ] \cdot 1000$$

wherein (⁴⁴Ca/⁴²Ca)ₛₐₘₚₗₑ is the ratio of the blood sample measured in the mass spectrometer and (⁴⁴Ca/⁴²Ca)_{standard} is the calcium isotope ratio of an internationally known and certified ratio. This approach allows an international comparability of the measured isotope ratios.

### Determination of the Threshold Values:

In a clinical study (Osteogeo study), statistically significant threshold values were ascertained for distinguishing women for δ^{44/42}Ca_{blood}, δ^{44/42}Caᵤᵣᵢₙₑ and (Ca/Sr)ᵤᵣᵢₙₑ within the scope of ROC (receiver-operating characteristics) analyses. In order to satisfy the definition of osteoporosis in accordance with the valid international (WHO World Health organisation) and national (DVA Dachverband für Osteoporose (Umbrella Association for Osteoporosis in Germany)), any study participants who had symptoms of vitamin D deficiency (≤25 μmol/l) had to be excluded. This affected 20 women, and therefore 80 women who satisfied the standard criteria for osteoporosis remained for the study.

Of these 80 women, 66 were characterised as having no signs of osteoporosis according to the X-ray result (DXA, gold standard), and 14 were diagnosed as suffering from osteoporosis.

TABLE 2
ROC-determined threshold values:

| | | | | L⁻; negative | L⁺; positive |
|---|---|---|---|---|---|
| | Threshold | Sensitivity | Specificity | likelihood | likelihood |
| | value (‰) | (%) | (%) | factor | factor |
| With Vitamin D | | | | | |
| deficiency* | | | | | |
| δ^{44/42}Ca_{blood} | −0.85 ± 10.06 | 100.0 | 55 | 0 | 2.2 (1.69-2.87) |
| δ^{44/42}Caᵤᵣᵢₙₑ | 0.16 ± 0.06 | 78.6 | 71.2 | 0.3 (0.11-0.83) | 2.73 (1.71-4.36) |
| [Ca/Sr]ᵤᵣᵢₙₑ | 2027 ± 250 | 63.3 | 75.8 | 0.47 (0.23-0.97) | 2.65 (1.49-4.73) |
| All | | | | | |
| δ^{44/42}Ca_{blood} | −0.85 ± 0.06 | 94.4 | 53.7 | 0.10 (0.02-0.70) | 2.04 (1.57-2.64) |
| δ^{44/42}Caᵤᵣᵢₙₑ | 0.23 ± 0.06 | 72.2 | 67.1 | 0.41 (0.19-0.89) | 2.19 (1.44-3.34) |
| [Ca/Sr]ᵤᵣᵢₙₑ | 1772 ± 250 | 72.2 | 61.0 | 0.46 (0.21-0.98) | 1.85 (1.25-2.75) |

Note:
*Vitamin D (≤25 μmol/l).

The negative likelihood factor (L⁻) specifies the likelihood for a value above the threshold value resulting in a positive osteoporosis diagnosis relative to a healthy person.

The positive likelihood factor (L⁺) specifies the likelihood for a value below the threshold value resulting in a positive osteoporosis diagnosis relative to a healthy person.

Exemplary Approach in the Diagnosis According to the Invention on the Basis of a Blood Sample (δ^{44/42}Ca_{blood}) and Urine Sample (δ^{44/42}Caᵤᵣᵢₙₑ).

Bearing in mind the great similarity of the values (Table 2), all 100 test subjects in the Osteogeo study were considered without limitation by the vitamin D concentration. Nevertheless, the sensitivity of the diagnosis increases when the vitamin D concentration is known (Table 2).

The statistical ROC analysis revealed, for the entire study group, a threshold value for δ^{44/42}Ca_{blood} of −0.85±0.06%; (δ^{44/42}Ca_{threshold value-blood}) The sensitivity of the method was calculated at 94.4%, and the specificity was 53.7% (Table 2). Blood-measured values of healthy test subjects lying above this threshold value have only a 0.1 times likelihood of resulting in a positive diagnosis (osteoporosis) (negative likelihood factor L⁻=0.1). Measured calcium isotope values below the threshold value have a 2.04 times higher likelihood of an ill person (positive likelihood factor L⁺=2.04) receiving a positive diagnosis (osteoporosis).

Exemplary Procedure in the Osteoporosis/Kidney Function Diagnosis According to the Invention for the Case that Both the Calcium Isotope Value of a Blood Sample (δ^{44/42}Ca_{blood}) and a Urine Sample (δ^{44/42}Caᵤᵣᵢₙₑ) are Present—FIG. 8:
a) forming the difference Δ_{urine-blood} and calculating the renal reabsorption (f_{renal-reabsorption}). Classifying the kidney function in accordance with Table 1.
b) δ^{44/42}Ca_{blood-measured}≥δ^{44/42}Ca_{threshold value-blood}+0.06. Blood-measured values of healthy test subjects that lie above this threshold value inclusive of the tolerance value have only a 0.1 times likelihood of resulting in a positive osteoporosis diagnosis. Green light! It is highly likely that osteoporosis is not present (green light!)
c) δ^{44/42}Ca_{threshold value-blood}+0.06 ≥δ^{44/42}Ca_{blood-measured}≥δ^{44/42}Ca_{threshold value-blood}−0.06%. Due to the position within the tolerance range of the threshold value, the patient is diagnosed with the onset of osteoporosis. Amber light! These patients are recommended for an additional DXA measurement in order to determine the individual risk of bone break for specific bones (for example femur)
d) δ^{44/42}Ca_{blood-measured}≤δ^{44/42}Ca_{threshold value-blood}−0.06%. Calcium isotope values measured in the blood below the threshold value inclusive of the tolerance value have a 2.04 times higher likelihood of a positive diagnosis (osteoporosis).

There is presumably a net loss of calcium and osteoporosis! These patients are recommended for an additional DXA measurement in order to determine the individual risk of bone break for specific bones.

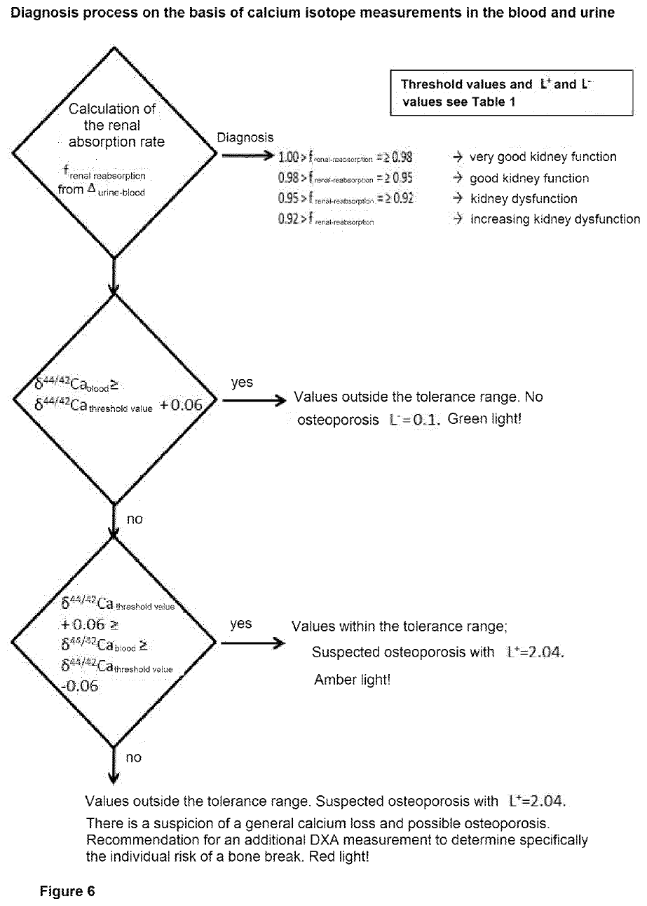

A graphical presentation of the diagnosis procedure is provided hereinafter: (see FIG. 6)

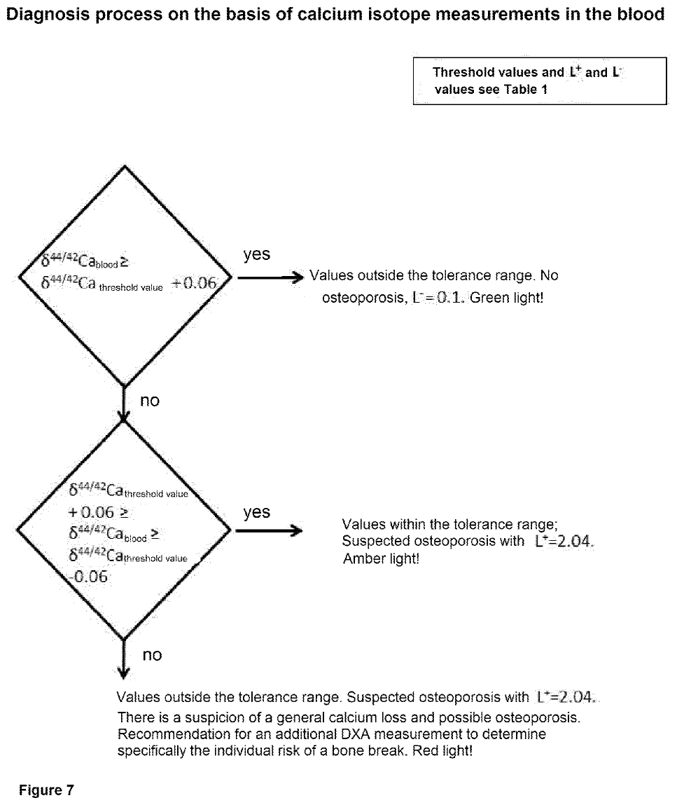

Exemplary Procedure in the Diagnosis According to the Invention for the Case that Only a Blood Sample (δ^{44/42}Ca_{blood}) is Present (FIG. 7):

A conclusion regarding osteoporosis can also be made if there is only a blood sample present. Due to the higher sensitivity of the blood samples as compared to the urine sample (Table 2), the diagnostic conclusion of a blood sample is higher than that of a urine sample, since the urine sample might be compromised by limited kidney function. The sensitivity of the diagnostic conclusion increases additionally if the vitamin D concentration of the patient is known (Table 2).
a) δ^{44/42}Ca_{blood-measured}≥δ^{44/42}Ca_{threshold value-blood}+0.06: Measured calcium isotope values above this threshold value inclusive of the tolerance value have only a 0.1 times likelihood of resulting in a positive diagnosis (osteoporosis). (Green light!)
b) δ^{44/42}Ca_{threshold value-blood}+0.06≥δ^{44/42}Ca_{blood-measured}≥δ^{44/42} Ca_{threshold value-blood}−0.06: Due to the position within the tolerance range of the threshold value, the patient is diagnosed with the onset of osteoporosis. (Amber light!) These patients are recommended for an additional DXA measurement in order to determine the individual risk of bone break for specific bones.
C) δ^{44/42}Ca_{blood-measured}≤δ^{44/42}Ca_{threshold value-blood -}0.06: Measured Ca isotope values below the threshold value inclusive of the tolerance value have a 2.04 times higher likelihood of a positive diagnosis (osteoporosis) (Red light!). There is a suspected general loss of calcium and possible osteoporosis. These patients are recommended for an additional DXA measurement in order to determine the individual risk of bone break for specific bones.
   Exemplary Procedure in the Diagnosis According to the Invention for the Case that Only a Urine Sample (δ^{44/42}Caᵤᵣᵢₙₑ) is Present (FIG. 8):

A conclusion regarding osteoporosis can also be made if there is only a urine sample present. The sensitivity of the diagnostic conclusion increases if the vitamin D concentration of the patient is known.
a) δ^{44/42}Ca_{urine-measured}≥δ^{44/42}Ca_{threshold value-urine}+0.06: Measured calcium isotope values above this threshold value inclusive of the tolerance value have only a 0.41 times likelihood of resulting in a positive diagnosis (osteoporosis). (Green light!)
b) δ^{44/42}Ca_{threshold value-urine}+0.06: ≥δ^{44/42}Ca_{urine-measured}≥δ^{44/42}Ca_{threshold value-urine}−0.06: Due to the position within the tolerance range of the threshold value, the patient is diagnosed with the onset of osteoporosis. (Amber light!) These patients are recommended for an additional DXA measurement in order to determine the individual risk of bone break for specific bones.
c) δ^{44/42}Ca_{urine-measured}≤δ^{44/42}Ca_{threshold value-urine}−0.06: Measured Ca isotope values below the threshold value inclusive of the tolerance value have a 2.19 times higher likelihood of a positive diagnosis (osteoporosis) (Red light!). There is a suspected general loss of calcium and possible osteoporosis. These patients are recommended for an additional DXA measurement in order to determine the individual risk of bone break for specific bones.

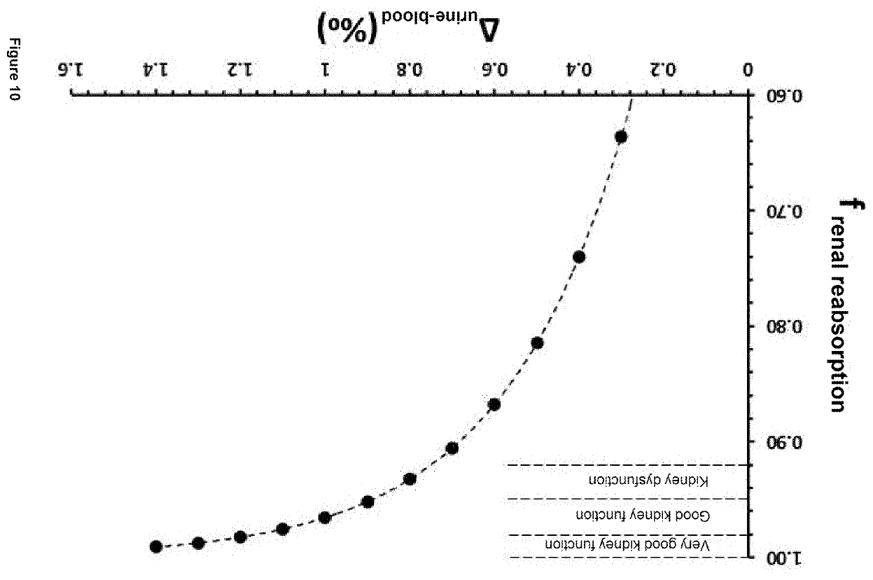

FIG. 9: Calcium isotope in the food, the stool, the blood and the urine in the post-menopausal women.

FIG. 10: Relationship between the isotopic difference between blood and urine (Δ^{44/42}Ca_{blood-urine}) and the rate of renal reabsorption (f_{renal-reabsorption}) as direct measure for kidney function. In addition, the defined ranges of different kidney functions (threshold values) are shown.

The invention also relates to a test kit for use in a method for diagnosing a disease that is accompanied by reduced bone density and/or calcium loss, comprising:
a packaging which comprises:
a collection container for receiving the sample liquid,
at least one monovette for receiving part of the sample liquid and for transporting it to a measuring device that determines isotope ratios and/or amount ratios of alkaline-earth metal portions, and
instructions, wherein the instructions have a fixed threshold value, which is independent of the individual, for the isotope ratio and/or the amount ratio of the alkaline-earth metal portions for comparison with the one or more sample values to be ascertained.

A fixed threshold value for the isotope ratio ⁴⁴Ca/⁴²Ca according to a δ notation in particular of δ^{44/42} Caᵤᵣᵢₙₑ=0.23±0.06% for a urine test kit or δ^{44/42} Ca_{blood}=−0.85±0.06% for a blood test kit can be specified in the instructions for the test kit.

Furthermore, the present invention relates to a test kit for use in a method according to the invention, comprising:
a packaging which comprises:
a collection container for receiving the sample liquid,
at least one monovette for receiving part of the sample liquid and for transporting it to a measuring device that determines isotope ratios and/or amount ratios of alkaline-earth metal portions, and
instructions, wherein a threshold value, which is independent of one or more factors determined by the individual patient, is specified in the test kit for comparison of the at least one sample value with the threshold value.

Such a test kit can comprise all of the features mentioned above for the method or all components necessary for the method according to the invention and its embodiments, individually or in combination.

An advantage of the test kit according to the invention is, in particular, that, for the first time, a single sample is sufficient to provide the basis for a diagnosis with respect to a disease accompanied by reduced bone density and/or calcium loss. A comparison with the fixed threshold value, which in particular is independent of the patient, constitutes a very significant simplification of the sequences performed with the previously known diagnosis kits.

1. A method for diagnosing a disease that is accompanied by reduced bone density and/or calcium loss, comprising the following steps:
a) determining isotope ratios or amount ratios of alkaline-earth metal portions in a sample of urine or blood or stool,
b) thereby determining at least one sample value, and
c) comparing the at least one sample value with a threshold value, characterised in that the threshold value is selected independently of one or more factors determined by the individual patient.

2. The method according to claim 1, wherein in step a) the isotope ratios or amount ratios of the alkaline-earth metal portions in the sample are determined by mass spectrometry.

3. The method according to claim 1, wherein in step a) the isotope ratios of calcium (Ca) are determined.

4. The method according to claim 1, wherein in step a) the amount ratio of the alkaline-earth elements of calcium (Ca) to strontium (Sr) are determined.

5. The method according to claim 1, wherein in step c) a fixed threshold value which is independent of the individual and which was defined beforehand depending on the sample type is used as comparison value.

6. The method according to claim 1, wherein the threshold value in step c) for the isotope ratio ⁴⁴Ca/⁴²Ca in a blood sample according to a δ notation is δ^{44/42}Ca_{blood}=−0.85±0.06%.

7. The method according to claim 1, wherein the threshold value in step c) for the isotope ratio ⁴⁴Ca/⁴²Ca in a urine sample according to a δ notation is δ^{44/42}Caᵤᵣᵢₙₑ=0.23±0.006%.

8. The method according to claim 6, wherein the threshold value, corresponding to formula:
$${δ}^{m 3 / m 2} Ca = {δ}^{m 3 / m 1} Ca \times [ \frac{{ln}^{( m 3 / m 2 )}}{{ln}^{( m 3 / m 1 )}} ]$$
with m3>m2>m1, wherein m3, m2, m1 represent the mass numbers, is transferrable to other isotope ratios of calcium.

9. The method according to claim 1, wherein the threshold value in step c) for the amount ratio of alkaline-earth elements of calcium (Ca) to strontium (Sr) is 1772±250 mol_{Ca}/mol_{Sr}.

10. The method according to claim 1, wherein in step b) one sample value is ascertained as δ^{44/42}Caᵤᵣᵢₙₑ and a further sample value is ascertained as δ^{44/42}Ca_{blood} and in step c) the difference between the two sample values is compared with a threshold value in order to additionally ascertain the kidney function.

11. The method according to claim 1, wherein in step b) one sample value is determined as δ^{44/42}Caₛₜₒₒₗ and a further sample value is determined as δ^{44/42}Ca_{blood} and in step c) the difference between the two sample values is compared with a threshold value in order to additionally ascertain the intestinal function.

12. The method according to claim 1, wherein a disease accompanied by reduced bone density and/or calcium loss is osteoporosis, osteomalacia, multiple myeloma and/or renal dysfunction.

13. The method according to claim 1, wherein the amount ratios in step a) are determined as molar ratios of the alkaline-earth metal portions in the sample or as mass ratios of the alkaline-earth metal portions in the sample.

14. The method according to claim 1, wherein if in step b) a sample value is determined as δ^{44/42}Caᵤᵣᵢₙₑ and in step c) the comparison with the threshold value according to a δ notation of δ^{44/42}Caᵤᵣᵢₙₑ=0.23±0.06% c reveals that the sample value is greater, the presence of an osteoporosis disease can be excluded as unlikely.

15. The method according to claim 1, wherein if in step b) a sample value is determined as δ^{44/42}Ca_{blood} and in step c) the comparison with the threshold value according to a δ notation of δ^{44/42}Ca_{blood}=−0.85±0.06% c reveals that the sample value is greater, the presence of an osteoporosis disease can be excluded as unlikely.

16. A test kit for use in a method for diagnosing a disease that is accompanied by reduced bone density and/or calcium loss, comprising:
a packaging which comprises:
a collection container for receiving the sample liquid,
at least one monovette for receiving part of the sample liquid and for transporting it to a measuring device that determines isotope ratios and/or amount ratios of alkaline-earth metal portions, and
instructions,
wherein the instructions have a fixed threshold value, which is independent of the individual, for the isotope ratio and/or the amount ratio of the alkaline-earth metal portions for comparison with the one or more sample values to be ascertained.

17. The test kit according to claim 16, wherein a threshold value for the isotope ratio ⁴⁴Ca/⁴²Ca according to a δ notation of δ⁴⁴Ca/⁴²Caᵤᵣᵢₙₑ=0.23±0.06% or δ⁴⁴Ca/⁴²Ca_{blood}=−0.85±0.06% is identified.

18. The test kit for use in a method according to claim 1, comprising:
a packaging which comprises:
a collection container for receiving the sample liquid,
at least one monovette for receiving part of the sample liquid and for transporting it to a measuring device that determines isotope ratios and/or amount ratios of alkaline-earth metal portions, and
instructions, wherein a threshold value, which is independent of one or more factors the individual, for comparison of the at least one sample value with the threshold value is specified in the test kit.
